(19) United States
(12) Patent Application Publication
(10) Pub. No.: US 2008/0288026 A1
(43) Pub. Date: Nov. 20, 2008

(54) Electro-Mechanical Connector for Thin Medical Monitoring Patch

(75) Inventors: Brett Cross, Seattle, WA (US); Steven C. Hugh, Bellevue, WA (US); Thomas A. Solosko, Issaquah, WA (US)

Correspondence Address: PHILIPS INTELLECTUAL PROPERTY & STANDARDS, 595 MINER ROAD, CLEVELAND, OH, 44143, US

(73) Assignee: KONINKLIJKE PHILIPS ELECTRONICS N. V., Eindhoven (NL)

(21) Appl. No.: 12/094,272

(22) PCT Filed: Oct. 30, 2006
(86) PCT No.: PCT/IB2006/054019

§ 371 Date: May 20, 2008

Related U.S. Application Data
(60) Provisional application No. 60/741,492, filed on Nov. 30, 2005.

(51) Int. Cl. A61N 1/08

(52) U.S. Cl. 607/60

(57) ABSTRACT

A device (8) comprises a medical device (10) which includes device electrical contacts (50). A patch (22) includes at least partially conductive first layer (28), which is in direct operative communication with a base surface (18). A patch connector (26) includes electrical contacts (42) being in electrical communication with the first conductive layer (28) and establishes an electrical communication path between the base surface (18) and medical device contacts (50), and a mechanical connection between the patch (22) and the medical device (10) when the patch connector (26) and the medical device (10) are engaged.

The following relates to the medical arts. It finds particular application in conjunction with medical monitoring devices and will be described with particular reference thereto. It will be appreciated that the following is also applicable to other medical and non medical devices in medical and non medical fields such as athletic monitoring, animal, child monitoring, electrical stimulation, medication delivery, and the like, in a variety of applications.

In many biomedical applications, monitoring and therapy devices are attached to the patient's skin to observe and monitor patient conditions such as health, blood flow, heart rhythm, blood oxygen levels, administer therapy as required, and the like. Examples of monitoring and therapy devices include the electrocardiograph to monitor ECG, external defibrillators, pacing devices, transcutaneous nerve stimulation devices, and transdermal drug delivery systems. In some applications, such as monitoring and therapy, devices are attached for extended periods of time and must be removed prior to showering, or when changing clothes. Many of the monitoring devices are large and typically are worn on a belt with wires attached to skin-mounted, disposable electrodes. The larger devices are uncomfortable to carry. Movement of the wires, monitoring device, or electrodes due to the attachment of wires may create artifacts in the monitored waveform.

Another approach is to use smaller external devices which are typically held in place with medical grade tape completely covering the device. Such method of skin attachment provides a secure and water-resistant adhesion to the skin, but does not allow the skin to breath or move under the device. Body moisture accumulates in occluded areas which aggravates the skin. Skin held rigidly under a device is affected by movements of the device. This creates artifact in the monitored waveforms. Rigidly held skin may also become irritated, especially at the tape-to-skin boundary. In addition, the constant pressure of the device against the skin may also cause depressions in the skin, which become irritating.

The present invention provides new and improved apparatuses and methods which overcome the above-referenced problems and others.

In accordance with one aspect, a skin-mounted device is disclosed. A medical device includes device electrical contacts. A patch includes a conductive first layer, which is in direct electrical communication with skin. A patch connector, which includes electrical contacts in electrical communication with the first conductive layer, establishes an electrical communication path between the skin and medical device contacts and affixes the medical device in close proximity to the patch.

In accordance with another aspect, a method of connecting to a subject base surface is disclosed. A patch, which includes a conductive first layer, is disposed in direct electrical communication with the base surface. The patch is electromechanically connected to a medical device connection interface which includes a first connector and device contacts.

In accordance with another aspect, a connector is disclosed which comprises a clip. The clip includes a first surface; a second surface opposite the first surface; extending portions disposed on the first surface; and a band of elastomeric material which extends from about the first surface to about the second surface to electrically contact device contacts of a monitoring device on the first surface and patch contacts on the second surface when the extending portions engage with a case of the medical device.

Still further advantages and benefits of the present invention will become apparent to those of ordinary skill in the art upon reading and understanding the following detailed description of the preferred embodiments.

The invention may take form in various components and arrangements of components, and in various steps and arrangements of steps. The drawings are only for purposes of illustrating the preferred embodiments and are not to be construed as limiting the invention.

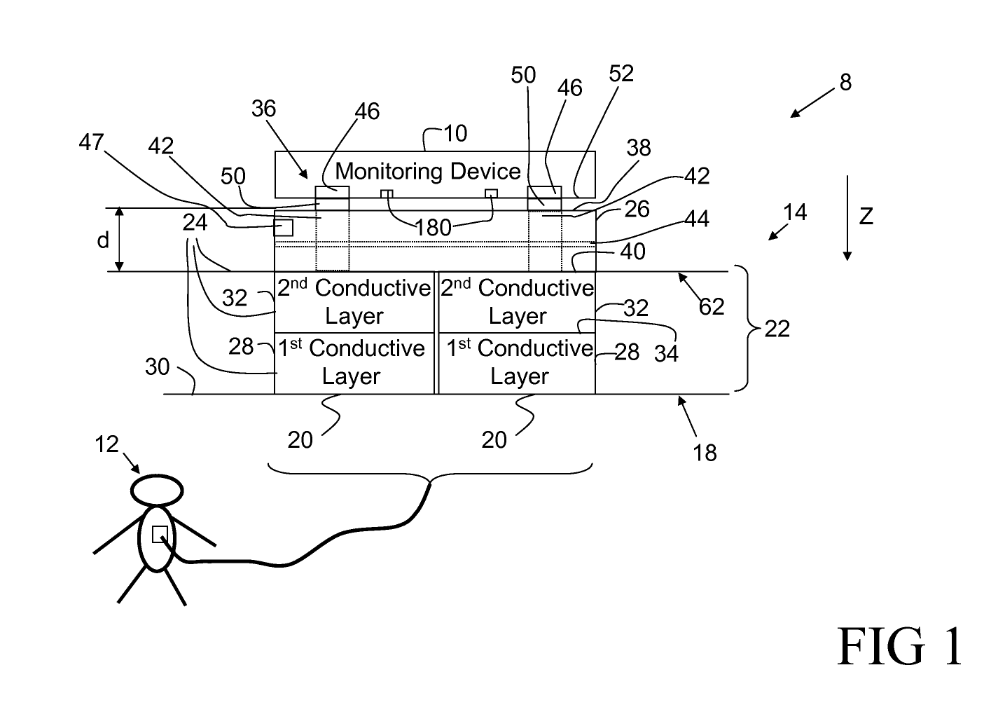

FIG. 1 is a diagrammatic illustration of a modular medical device;

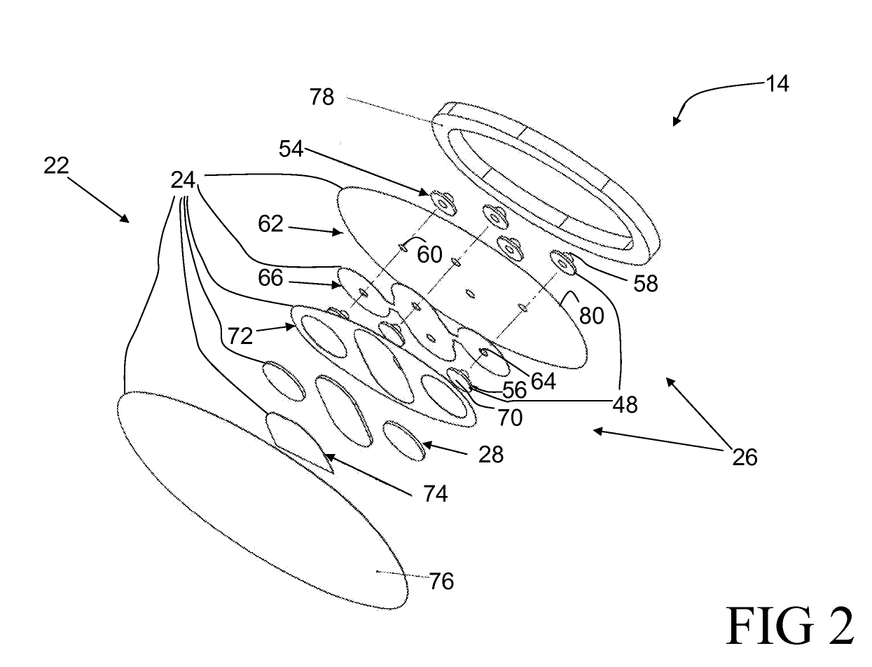

FIG. 2 is an expanded view of a patch assembly;

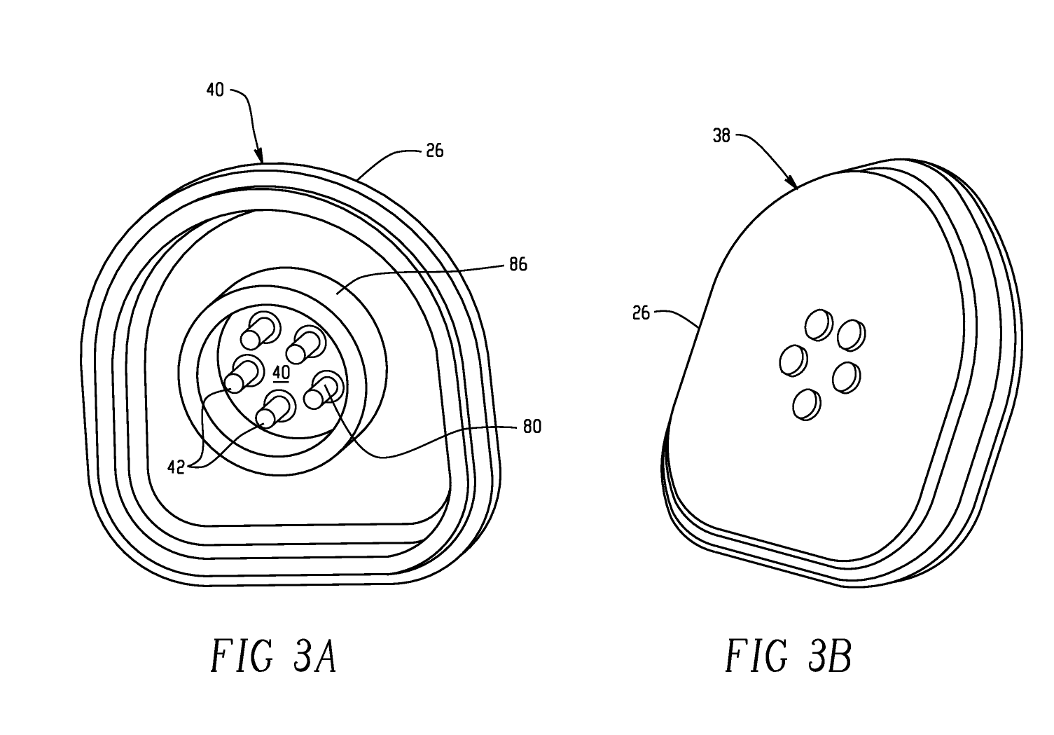

FIG. 3A is a perspective view of one face of a connector which includes pins;

FIG. 3B is a perspective view of an opposite face of the connector of FIG. 3A;

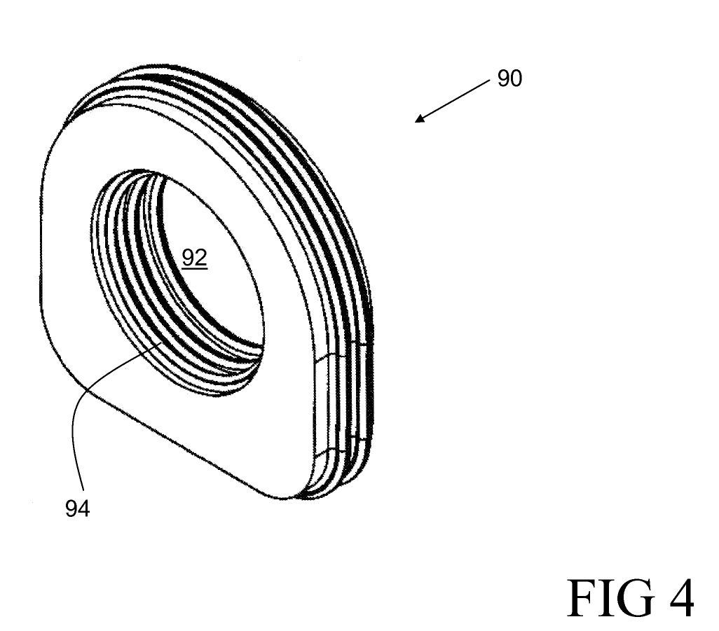

FIG. 4 is a perspective view of a portion of a mechanical interface of connector of FIG. 3A;

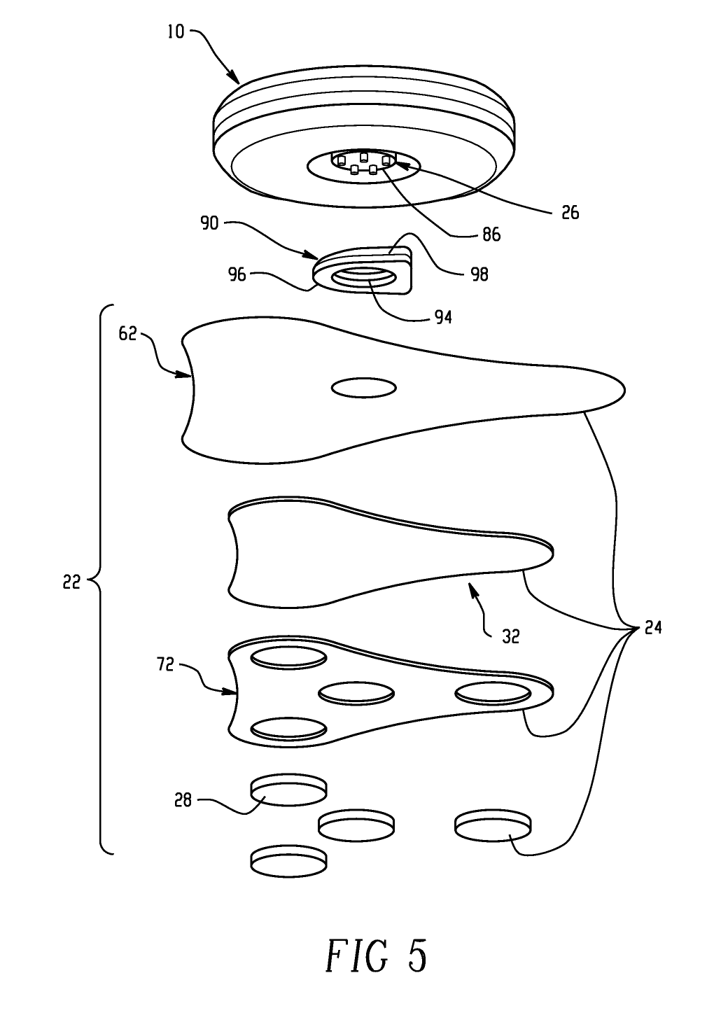

FIG. 5 is an expanded view of the monitoring device and the patch assembly;

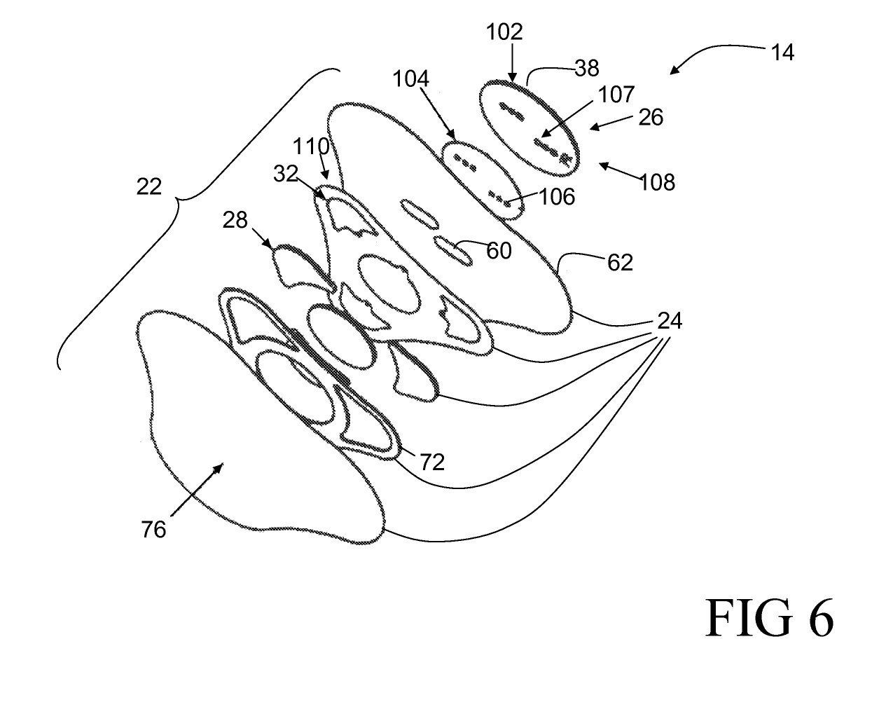

FIG. 6 is an expanded view of a patch assembly which includes an electronic board;

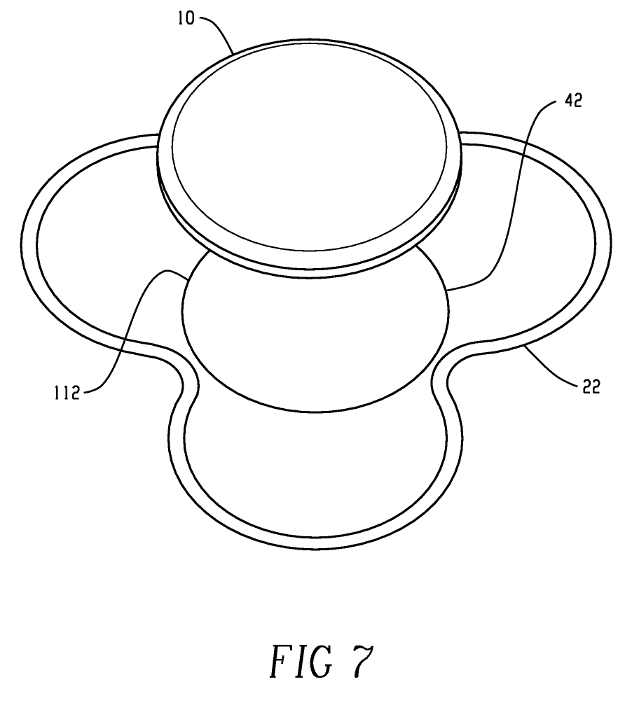

FIG. 7 is a view of the monitoring device and the patch which are connected via a single piece of Z-axis conductive pressure sensitive adhesive;

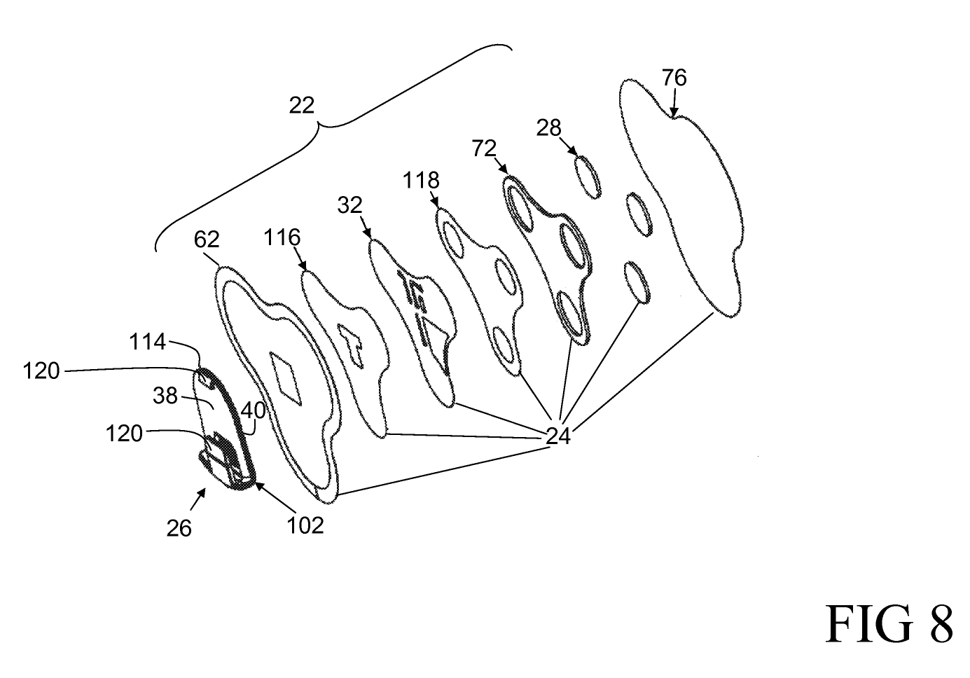

FIG. 8 is an expanded view of a patch assembly which includes a clip;

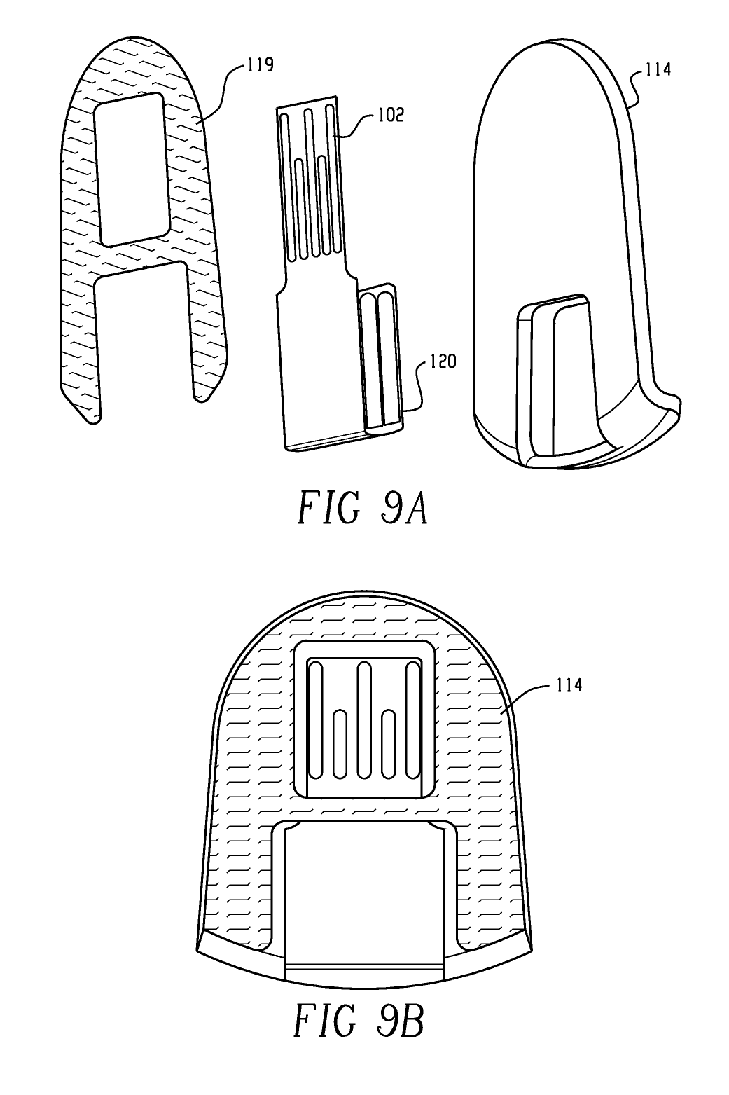

FIG. 9A is an expanded view of a clip assembly;

FIG. 9B is a diagrammatic illustration of the assembled clip of FIG. 9A;

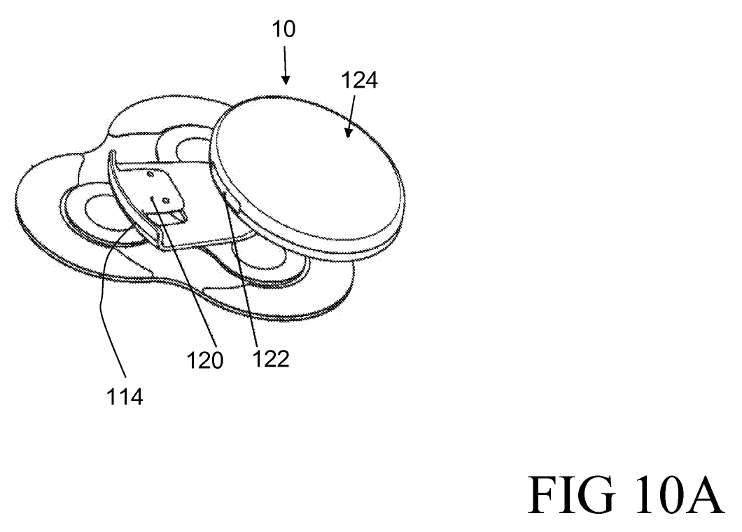

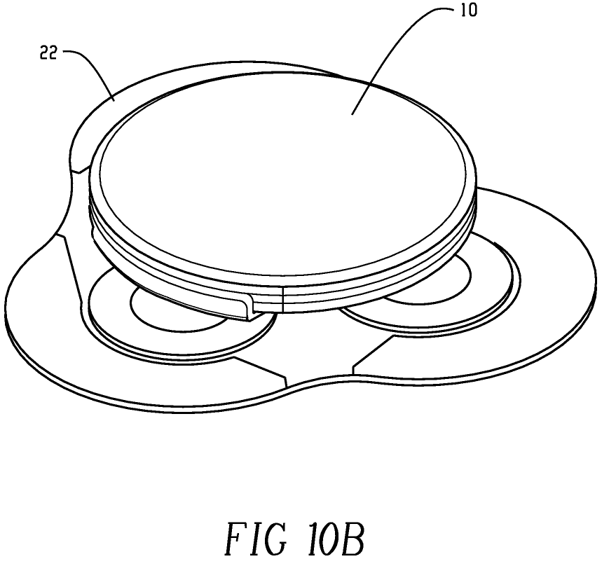

FIGS. 10A and 10B are diagrammatic illustrations of an assembly of the patch assembly of FIG. 8 and a monitoring device;

FIGS. 11A, 11B and 11C are diagrammatic illustrations of the monitoring device which snaps completely into the clip;

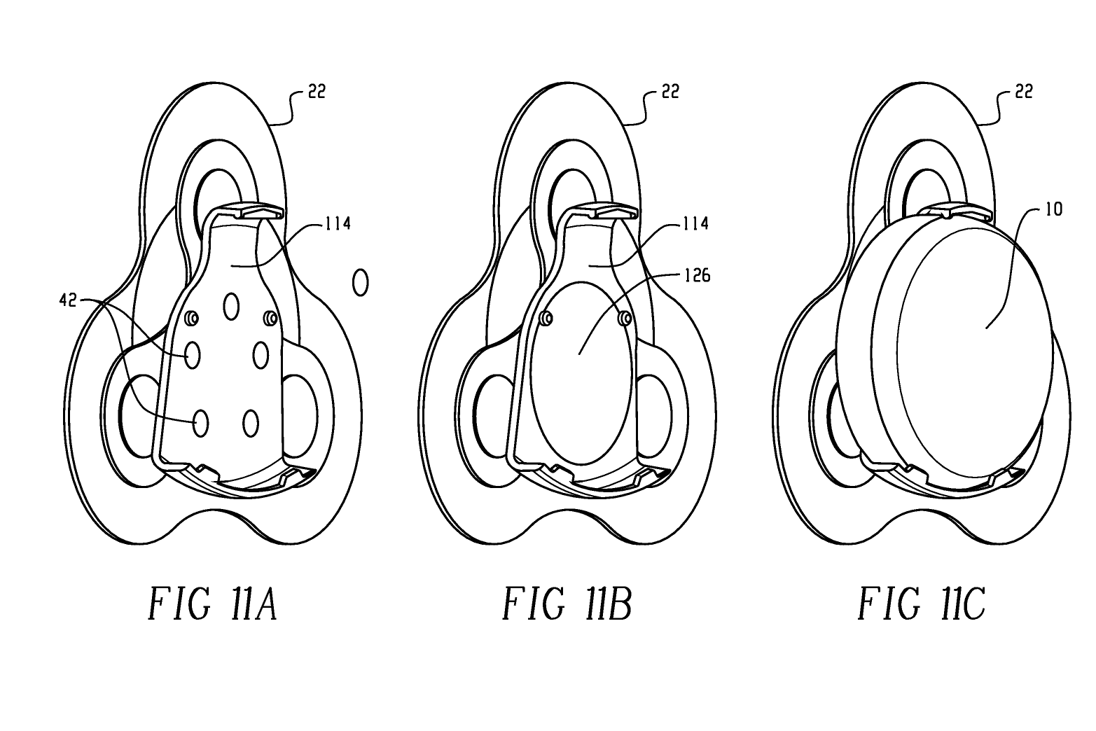

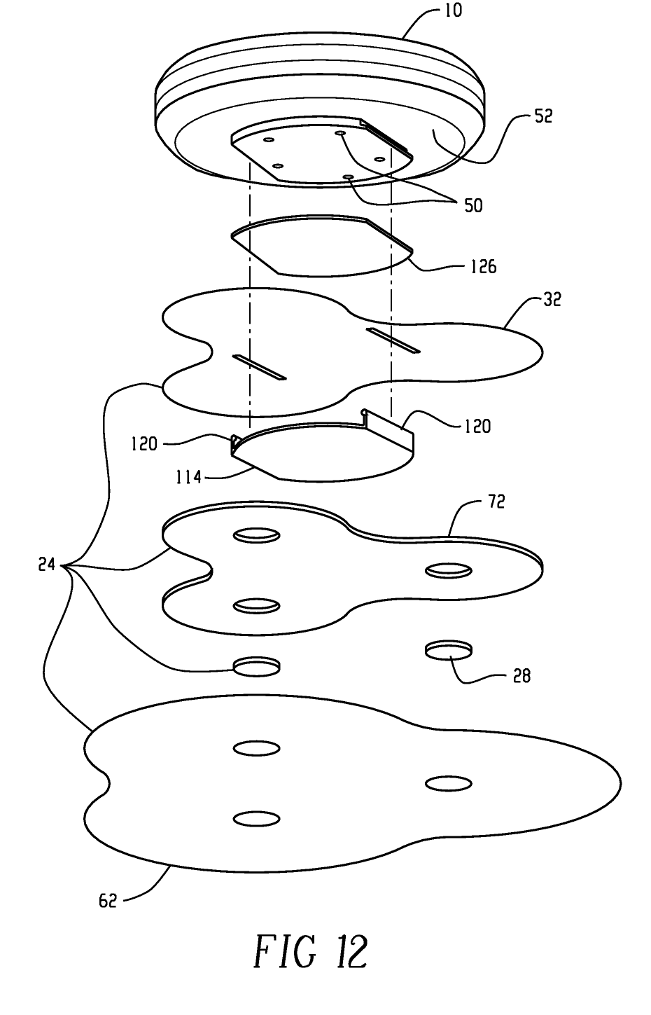

FIG. 12 is an expanded view of an assembly of the patch assembly, in which a clip is inserted through the patch layers, and a monitoring device;

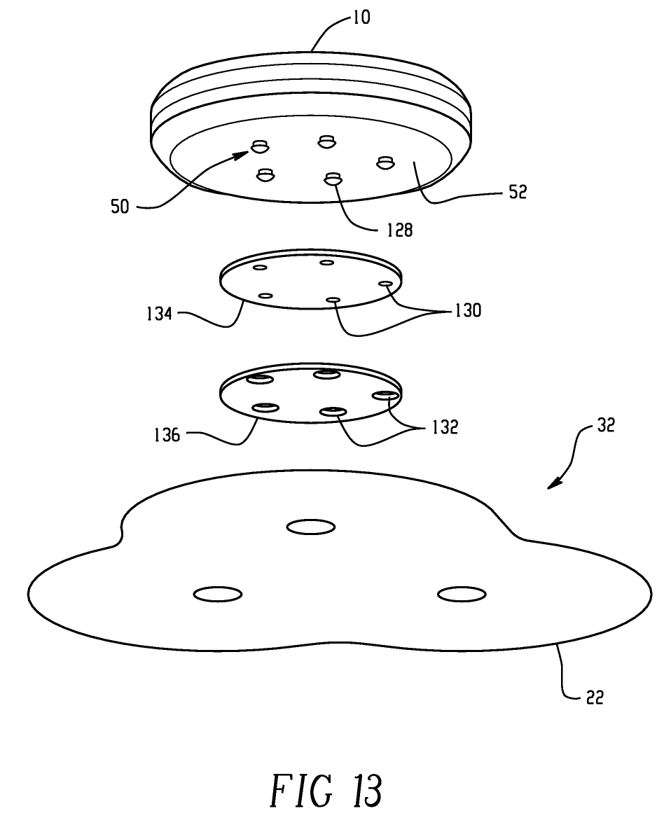

FIG. 13 is an expanded view of an assembly of a patch and a monitoring device;

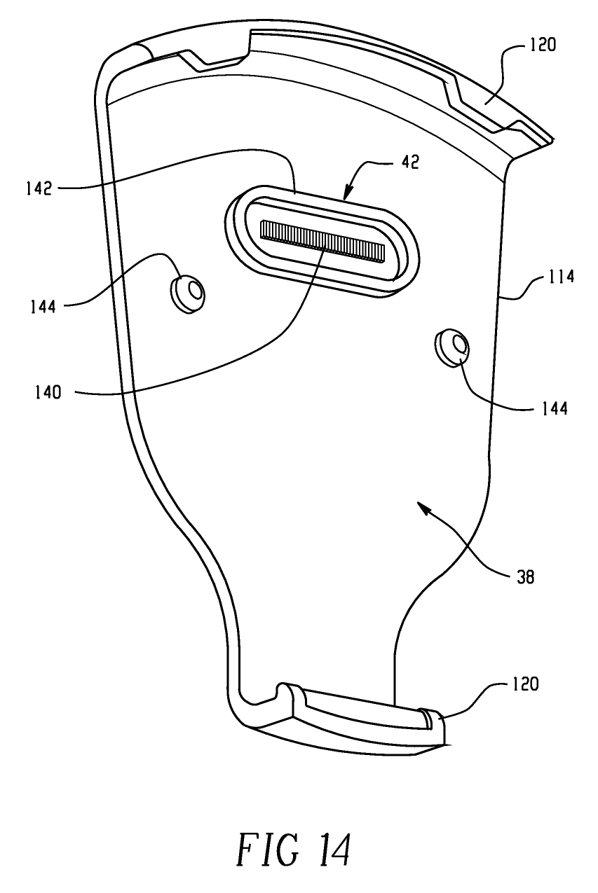

FIG. 14 is a diagrammatic illustration of one side of a clip which includes a single silicon band;

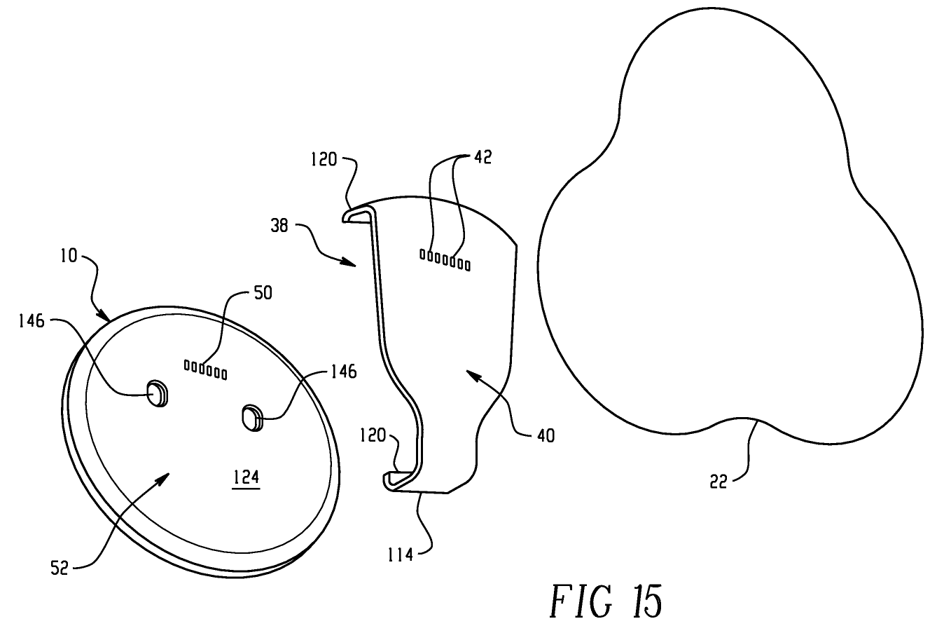

FIG. 15 is an expanded view of an assembly of a monitoring device, patch and clip of FIG. 14;

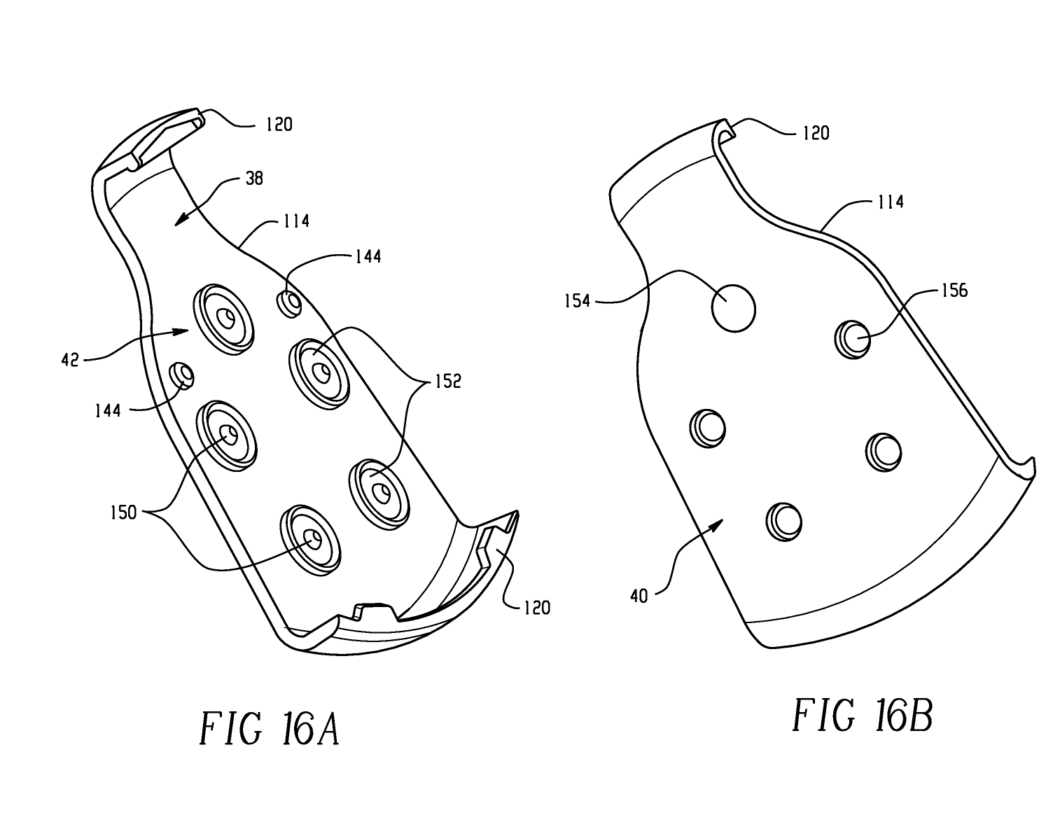

FIG. 16A is a perspective view of one face of a clip;

FIG. 16B is a perspective view of an opposite face of the clip of FIG. 16A;

FIG. 17A is a perspective view of one side of a clip;

FIG. 17B is a perspective view of an opposite side of the clip of FIG. 17A;

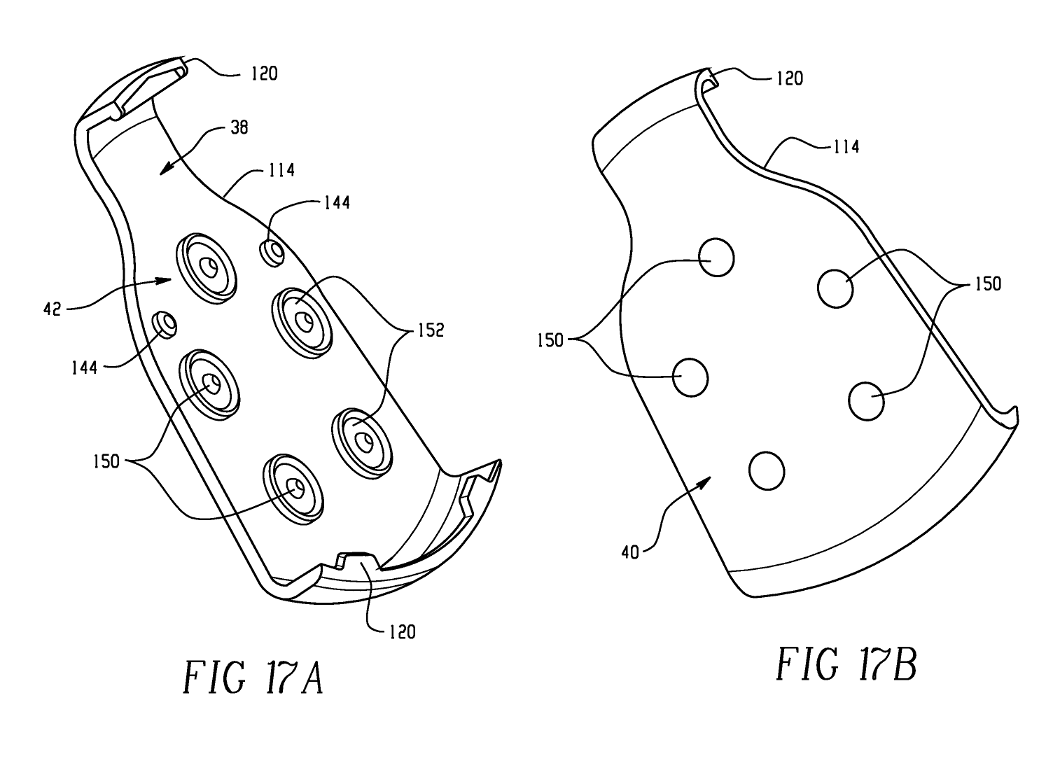

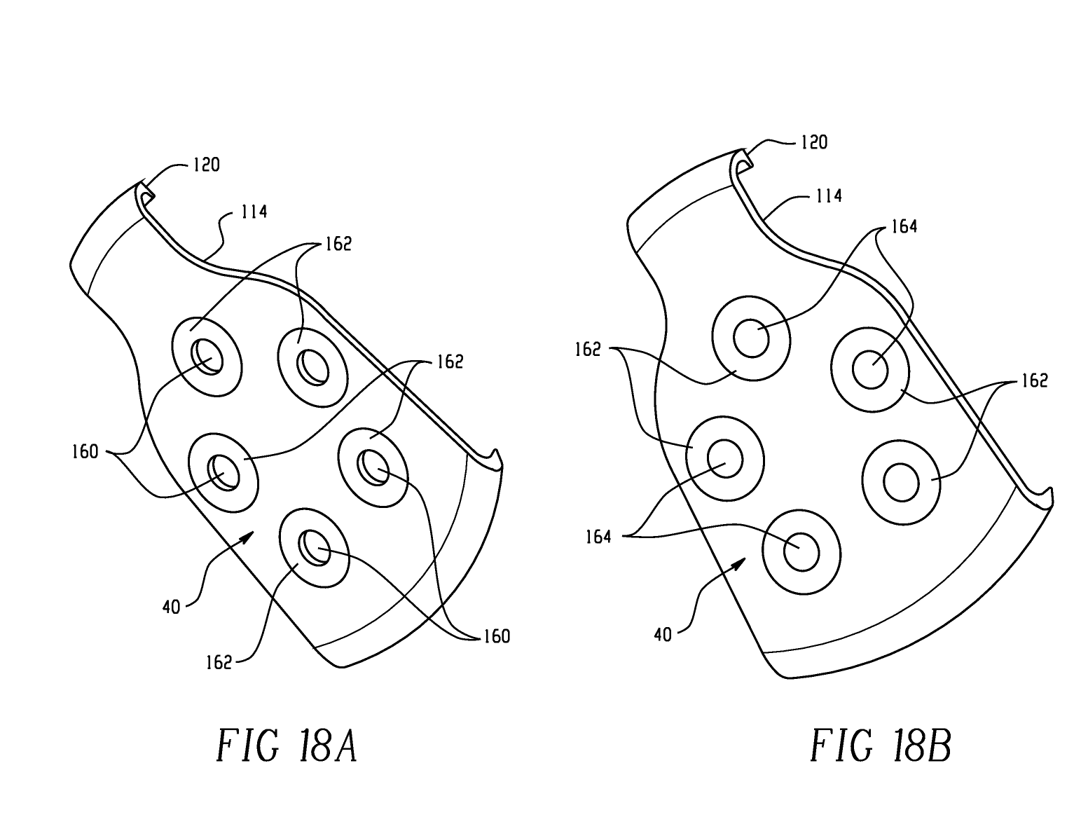

FIG. 18A is a perspective view of a portion of a clip;

FIG. 18B is a perspective view of the clip of FIG. 18A with overmolded inserts;

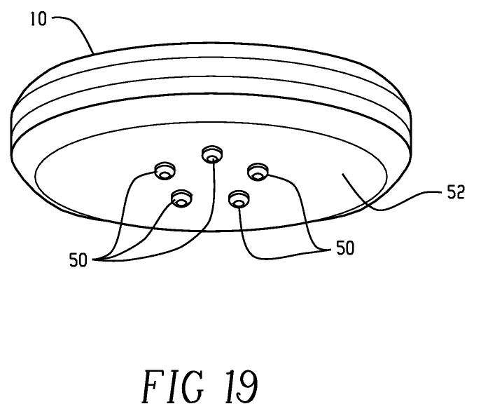

FIG. 19 is a diagrammatic illustration of the monitoring device which includes reusable conductive posts;

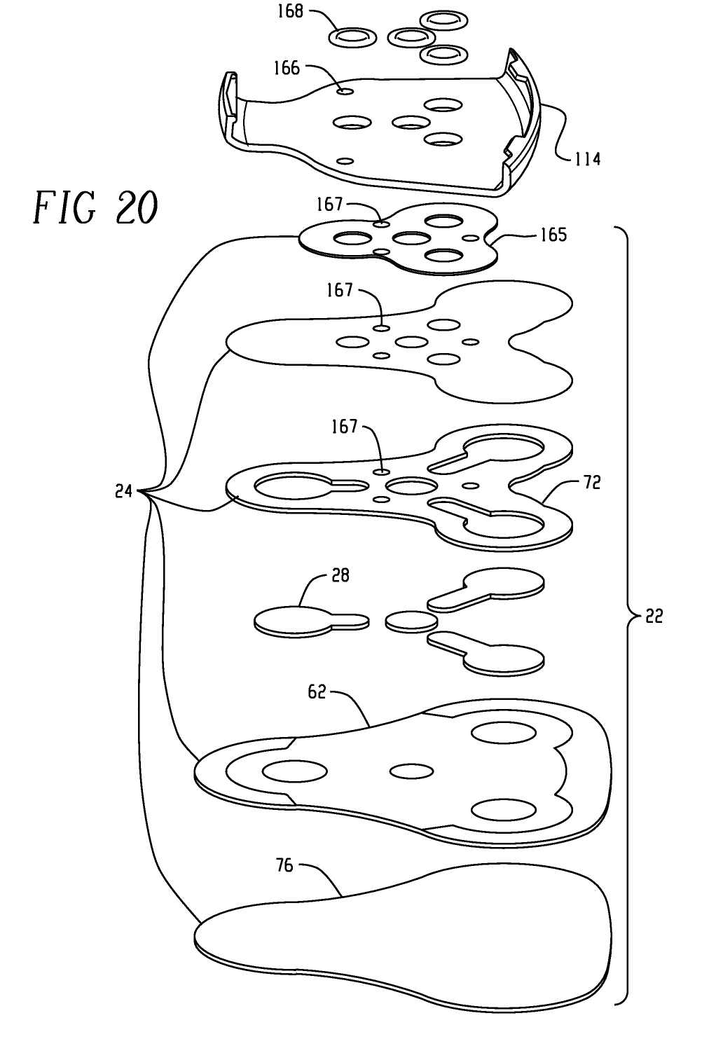

FIG. 20 is an expanded view of an assembly of a patch and a monitoring device of FIG. 19;

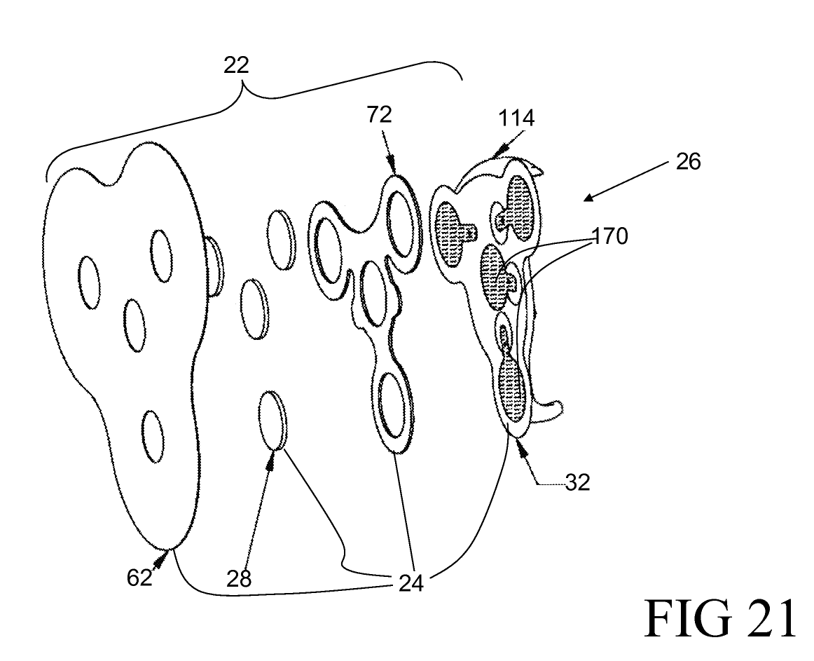

FIG. 21 is an expanded view of the clip molded directly onto the circuit layer;

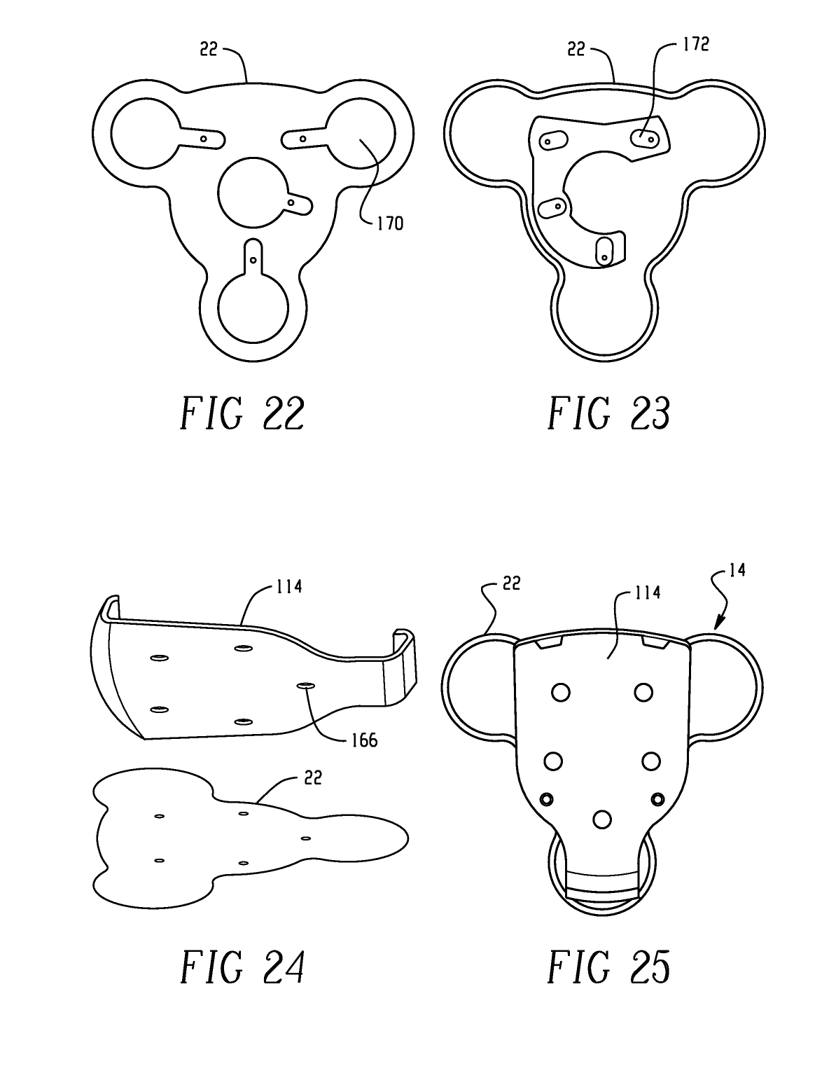

FIG. 22 is a perspective view of the patch of FIG. 21 with a patch side printed circuit;

FIG. 23 is a perspective view of the patch of FIG. 21 with a clip side printed contact pads;

FIG. 24 is a perspective view of the clip and the patch of FIG. 21; and

FIG. 25 is a perspective view of the assembled clip and patch of FIG. 21.

With reference to FIG. 1, a modular device 8 includes a monitoring or therapy or other medical device 10 which is attached to a patient 12 via a patch assembly 14. The patch assembly 14 provides a communication path between the monitoring device 10 and a base surface or skin 18 of the patient 12 in one or more areas 20. More specifically, the patch assembly 14 includes a patch or patch laminate 22 including a plurality of layers 24 and an electromechanical connector or connection interface 26. In one embodiment, a thickness d of the electromechanical interface 26 is less than 10 mm. Examples of medical devices 10 are cardiac ECG event monitors, ECG Holter recorders, and cardiac emergency alerting devices.

A first conductive material layer or pieces of first conductive material or first electrodes 28, such as conductive hydrogel, are disposed at a proximate or first surface 30 of the patch assembly 14 to make direct contact with the skin 18. A second layer or electrodes or patch circuit layer 32 is disposed proximately to a top surface 34 of the first conductive hydrogel layer 28. In one example, the second electrodes 32 are constructed from silver/silver chloride (Ag/AgCl) or from any other suitable material. In one embodiment, the electrodes 32 are disposed in communication with the skin 18 to measure the voltage difference between two or more locations on the body. Although only two patch contacts 32 are illustrated, it is contemplated that a number of contacts may be greater than two.

The monitoring device 10 includes a connection interface 36. The patch connector 26 is disposed proximately to the monitoring device 10 at a connector first or top layer or surface 38 and to the patch assembly 14 at a connector second or bottom layer or surface 40. Ionic conduction from the skin 18 passes through the first conductive or hydrogel material layer 28, changes to electronic conduction in the circuit layer 32 and as such is passed to the monitoring or therapy device 10 via electrical contacts or connection interface 42 of the electromechanical connector 26. As discussed in detail below, in one embodiment, the patch connector 26 is a stand alone connector which is disposed between the monitoring device 10 and the patch laminate 22. In one embodiment, the patch connector 26 includes connection interfaces disposed throughout the patch layers 24. In one embodiment, the connector 26 includes a rigid base layer 44 and/or a mechanical member or members 47 to establish a rigid mechanical connection between the patch 22 and the monitoring device 10.

The first conductive hydrogel layer 28 improves the electrical conductivity between the electrodes 32 and the skin 18. Typical components of a conductive hydrogel include water, which acts as the solvent, water-soluble monomers, which crosslink to give structure to the gel and which may also provide skin adhesion, humectant materials which reduce the dryout characteristics of the hydrogel material, and electrolytes or salts such as sodium chloride or potassium chloride dissolved in water, which provide and facilitate ionic movement and conductivity. One advantage of hydrogel materials over other conductive electrolytes is that the hydrogel material can be removed cleanly from the skin without leaving a residue

The electromechanical connector 26 provides the electrical connections between the patch circuit layer 32, which is in electrical contact with the skin 18 via the first conductive layer 28, and the monitoring or therapy device 10. The electrical connections may either be low impedance connections, such as a pin-to-metal pad connection, or high impedance connections, such as a higher-impedance conductive silicone to metal pad connection. The electromechanical connector 26 also provides the mechanical connection to the monitoring device 10 via a monitoring device connection interface or second or device connector 46. The methods of mechanical connection as well as the type of electrical connection, either high or low impedance, play a role in reducing artifact in the monitored signal as described below.

Electrical artifacts include artifacts due to common mode voltages and electrostatic charges that affect the skin, electrode patch and monitoring device. Motion artifacts include voltages produced by the skin, fat and muscle during skin stretching under the electrodes, movement of the electrode or sensor on the skin, intermittent electrode or sensor contact with the skin, wire movement, and movement of the monitoring or therapy device which translates to movement of electrodes or sensors on the skin. In addition, light sensors are also subject to artifact from external light sources.

Rigid mechanical connections can drive the artifact frequency band higher, while, looser, floppy mechanical connections can dampen artifacts and drive the artifacts to a lower frequency. Rigid patch materials may hold the skin tighter and reduce the amplitude of artifacts caused by skin and muscle stretching. Decoupling the electrodes from each other and from the monitoring or therapy device may decrease the affect of device movement on the electrodes.

With continuing reference to FIG. 1 and further reference to FIG. 2, the second electrodes 32 are omitted. The patch connector 26 provides low impedance electrical connection of the patch assembly 14 to the medical device 10. More specifically, the electrical contacts 42 of the patch connector 26 include snaps 48 which connect to device contacts 50 which are disposed about a bottom surface 52 of the monitoring device 10. The snaps 48 provide electrical and mechanical connection of the patch assembly 14 to the monitoring device 10. More specifically, each snap 48 includes a snap top 54 and a snap eyelet 56. Each snap top 54 includes a snap post 58 which is inserted into respective matching snap receptacle (not shown) in the monitoring device 10 to hold the snaps 48 rigidly in place. Three, four, five, six or more snaps 48 form a stabilizing plane to hold the monitoring device 10 rigidly against the patch assembly 14 and distally from the base surface or skin 18.

Each snap top 54 connects with respective snap eyelet 56 through corresponding openings 60 in a retention seal layer 62, and openings 64 in a snap support layer 66 to establish electrical contact. The retention seal layer 62 protects the hydrogel layer 28 from outside water entry; while the snap sealing layer 66 prevents the snaps 48 from tearing out of the patch assembly 14. The snap sealing layer 66 is constructed from polyester or other appropriate stiff supporting material. In one embodiment, the snap eyelets 56 are constructed from a conductive material. In another embodiment, each snap eyelet 56 is coated with a conductive material such as silver/silver chloride to provide low offset and low noise body signal (ECG or other) measurements. A first face or side 70 of each snap eyelet 56 makes contact with the first conductive layer 28 such as pieces of hydrogel material which make contact with the base surface or skin 18. The first pieces of hydrogel material 28 are disposed in a hydrocolloid or frame layer 72

A non-conductive liquid-proof sealing layer 78 between the monitoring device 10 and the patch assembly 14 surrounds the snaps 48 to provide additional protection for the snaps 48 from outside liquids such as shower water. In one embodiment, the sealing layer 78 includes a single, compressible, elastomer gasket that is bonded to a top surface 80 of the retention seal layer 62 which compresses when the monitoring device 10 snaps onto the snaps 48. In one embodiment, the height of the uncompressed gasket is greater than the snap posts 58. The gasket compresses and creates the seal as the snaps mate with the respective mating receptacles. Individual discrete seals may be used around each snap 48 in place of the single elastomer gasket.

With continuing reference to FIG. 1 and further reference to FIGS. 3A and 3B, the patch assembly 14 in this embodiment includes the patch connector 26 which is a molded connector which includes individual connector contacts 42 such as individual pogo-style spring-loaded pins 80 to provide a low impedance connection. The pins 80 are post-inserted or insert molded into the connector through the top or first surface 38 of the connector 26 (as seen in FIG. 3B) to allow the spring-loaded end of each pin 80 to extend through the second or bottom surface 40 of the connector 26 into a sealing boss 86. The sealing boss 86 is drafted inwards to provide mechanical locking feature for mechanical connection with the monitoring device 10. More specifically, a top surface of the sealing boss 86 which is distal from the bottom surface 40 of the connector 26 is larger than a rear surface of the sealing boss 86 which is proximate to the bottom surface 40 of the connector 26.

With continuing reference to FIGS. 3A and 3B and further reference to FIGS. 4 and 5, a non-conductive elastomeric sealing boot 90, which includes an opening 92 with ribbed walls 94 in a central portion, is connected to the connector 26. More specifically, when the connector 26 and the sealing boot 90 are mated, the rigid sealing boss 86 pushes through the ribbed walls 94 of the center opening 92 of the sealing boot 90, compressing the elastomer material and creating radial force which holds the patch 22 via the boot 90 onto the connector 26. To reinforce, the inward draft on the walls of the sealing boss 86 prevents the sealing boot 90 from sliding off. Since the compressed elastomer wants to relax, the effect of the inward draft causes the boot 90 to slide inward, towards the inside of the connector 26, further trapping and holding the boot 90 and the patch 22 against the connector 26. Outside walls 96 of the sealing boot 90 include ribs 98 which compress and deflect against the inner walls of the connector 26 to form a seal against outside moisture entry into the boss 86 of connector 26.

Once the sealing boot 90 is mated with the connector 26, the spring-loaded pins 80 contact metal pads or traces provided on the patch circuit layer 32 of the patch 22. The pads or traces lead to the first individual conductive pieces 28 and carry signals from the first pieces 28 to the monitoring device 10 and from the monitoring device 10 to the first pieces 28. For example, the pads and traces may be printed using silver, silver/silver chloride, or conductive carbon ink on a polyester or PVDF substrate. As another example, the pads and traces may be plated copper traces on a flexible polyester or Kapton substrate. As yet another example, the pads and traces may be part of a printed circuit board.

With reference again to FIG. 1 and further reference to FIG. 6, the electrical contacts 42 of the patch connector 26 of this embodiment include a connector printed circuit layer or board 102, which is bonded to the retention seal layer 62 via a die-cut piece of a non-conductive pressure sensitive adhesive layer (PSA) 104. The retention seal layer 62 and pressure sensitive adhesive layer 104 include respective openings 60, 106 to be filled with conductive epoxy 107 which electrically connects the patch circuit layer 32 to conductive pads 108 disposed on the printed circuit board 102. More specifically, during the assembly, the pressure sensitive adhesive layer 104 is aligned over printed circuit pads 110 on the patch circuit layer 32, which for example is, a printed polyester layer, and adhered to the retention seal layer 62. Each of the openings 106 in the PSA layer 104 is filled with conductive epoxy. The connector printed circuit board 102 is aligned with the PSA layer 104. The entire patch assembly 14 is placed in a heated chamber to hasten the cure of the conductive epoxy. Alternatively, since the conductive epoxy is trapped between the circuit board 102 and the patch circuit layer 32, the conductive epoxy may be allowed to cure at room temperature as the patch assembly 14 is packaged and shipped.

In one embodiment, the top surface 38 of the connector 26, includes a multi-pin straight connector or right angle header with or without a housing snap which enables the electromechanical attachment of the patch assembly 14 to the monitoring device 10. In one embodiment, the sealing gasket is bonded to the top surface 38 of the connector 26. Upon insertion of the connector 26 into the monitoring device 10, the gasket compresses to protect the connector pins from liquid entry.

With continuing reference to FIG. 1 and further reference to FIG. 7, the patch connector electrical connection interface 42 includes a single piece or layer 112 of Z-axis conductive pressure sensitive adhesive (PSA), which directly electrically connects the patch electrodes 32 to the device contacts 50 on the bottom surface 52 of the monitoring device 10. To establish a reliable connection, the Z-axis conductive PSA is pressed between the top surface 38 of the patch assembly 14 and the bottom surface 52 of the monitoring device 10 with a sufficient amount of pressure for a sufficient amount of time, which is specific to the individual material. For example, a pressure of 30 PSI which is applied for 5 seconds may be sufficient for 3M 9703™, but may not be sufficient for a different PSA. Many conductive PSA materials are not suitable for use alone as structural PSA layers. A strengthening, non-conductive PSA layer is applied around such conductive PSA to improve strength and support, and to bring the Z-axis conductive PSA into compression which provides a more consistent electrical connection.

With continuing reference to FIG. 1 and further reference to FIGS. 8, 9A and 9B, the patch assembly connector 26 of this embodiment includes a clip 114. The clip 114 includes the top surface 38 proximate the monitoring device 10, bottom surface 40 proximate the patch 22 and the connector printed circuit layer 102, such as a flexible circuit layer, which mates with the patch circuit layer 32, which, in this embodiment, is a flexible circuit layer disposed between first and second dielectric layers 116, 118. The connector flexible circuit layer 102 is aligned so that its traces mate with respective traces of the patch circuit layer 32. To improve electrical connection between the two sets of traces, a low-impedance Z-axis conductive adhesive is applied to the connector flexible circuit 102 which bonds the connector printed circuit 102 to the patch circuit 32 and electrically connects the traces. The clip 114 includes a non-conductive layer of pressure sensitive adhesive 119 which bonds the clip 114 to the patch 22 mechanically and structurally. The clip non-conductive PSA layer 119 provides the liquid-proof seal between the clip 114 and the patch 22.

With continuing reference to FIGS. 1 and 8 and further reference to FIGS. 10A and 10B, a tongue or extending portion 120 of the clip 114 is inserted into a slot 122 of a case 124 the monitoring device 10. Once inside the monitoring device 10, the traces on the bottom surface of the clip tongue 120 mate with the device contacts 50 in the monitoring device 10. This completes the electrical circuit between the patch traces and the monitoring device 10.

With continuing reference to FIGS. 1 and 8 and further reference to FIGS. 11A, 11B and 11C, the monitoring device 10 snaps completely into the clip 114, which is, for example, a snap action clip. As a result of such mechanical connection, the device contacts 50, such as pins and leaf springs, on the bottom surface 52 of the monitoring device 10 connect electrically with corresponding patch metal contacts 42, i.e., gold-plating over nickel-plated copper pins, or the connector printed circuit board 102 of the clip 114 through a single piece of thin, Z-axis conductive elastomer sheet 126. The elastomer sheet 126 compresses as the clip 114 snaps onto the monitoring device 10. Once compressed, the conductive particles, i.e. carbon particles or fibers, are pressed against conductive surfaces of the clip and monitoring device making a connection. In addition, since the elastomer sheet is conductive in the Z-axis only, such elastomer sheet forms a seal against outside moisture entry when compressed. The patch 22 is attached to the clip 114 with one or more pieces of pressure sensitive adhesive. A conductive adhesive, such as 3M 9703™, can be used to make the electrical connection between the patch traces and the metal contacts in the clip 114. A piece of non-conductive structural adhesive may be used around the conductive PSA layer to improve the strength and reliability of the bond between the patch 22 and clip 114.

With reference to FIG. 12, to eliminate the conductive adhesive between the clip 114 and the patch 22, the clip 114 includes one or more extending portions 120 which are inserted through the patch circuit layer 32. The clip 114 is held in place by the frame layer 72. The patch connector electrical connection interface 42 includes the layer 126 of a Z-axis conductive elastomer material which provides electrical connection between the monitoring device 10 and the patch circuit layer 32. The patch traces are directly exposed to the Z-axis conductive silicone layer 126, which electrically connects the patch traces to the device contacts 50 on the bottom surface 52 of the monitoring device 10. Such direct connection is more reliable and robust, since every additional electrical interface increases the risk for the patch failure. By passing under and through the patch circuit 32 and mechanically by snap action connecting with the monitor 10, the clip 114 provides a rigid support surface against which the monitoring device 10 may press into the patch traces. The rigid surface provides a more consistent pressure between the monitoring device and patch contacts.

With reference to FIG. 13, the device contacts 50 of the monitoring device 10 include pins which make direct contact with the patch circuit layer 32. The monitoring device pins 50 each includes a shoulder 128, which tapers to a point near the end. The pins 50 press through or make openings 130, 132 in respective first or top and second or bottom layers 134, 136 of a non-conductive elastomer. The top layer openings 130 have smaller diameter than the bottom layer openings 132. Further, the diameter of each top layer opening 130 is smaller than the dimensional measurements of the shoulder 128 of the pins 50. The top layer opening 130 traps respective shoulder 128 of each pin 50 once the pins are pressed through the openings 130, 132 and force the pins 50 to contact exposed pads or traces on the patch circuit layer 32.

With reference again to FIG. 1 and further reference to FIGS. 14 and 15, the patch connector electrical connection interface 42 includes a single band of higher-impedance Z-axis conductive elastomer 140, such as silicone, surrounded by a single, conductive or non-conductive elastic seal 142. When compressed, the conductive silicone 140 provides electrical connection between the device contacts 50 on the bottom surface 52 of the monitoring device 10 and conductive traces on the patch 22. For example, the conductive silicone 140 can be inserted or over-molded to pass completely through the clip 114 and make contact with the monitoring device 10 on the clip top surface 38 and the conductive traces or pads of the patch 22 on the clip bottom surface 40. E.g., no additional clip contacts are required. Since the conductive silicone 140 electrically connects the patch traces to the device contacts 50 on the monitoring device 10, non-conductive PSA may be used to bond the patch layers to the clip 114. Although, the device contacts 50 of the monitoring device 10 are shown to be flush with the bottom surface 52 of the monitoring device 10, it is contemplated that the device contacts 50 of the monitoring device 10 can extend beyond the bottom surface 52 of the monitoring device 10 or be recessed into the bottom surface 52 of the monitoring device 10. The clip extensions 120 releasably engage the case 124 of the medical device 10 to hold the medical device 10 and the patch 22 together.

To ensure enough compression of the conductive silicone, the patch traces may be backed up with another material, such as a thicker, firmer material, e.g. 0.032 inch-thick polyethylene foam.

The elastic seal 142 surrounds the single piece or array of conductive silicone element(s) and, when compressed against the base of the monitoring device, prevents liquid entry into the contact area.

First alignment structures or means 144 disposed on the first clip surface 38 mate with second alignment structures or means 146 disposed on the bottom surface 52 of the monitoring device 10 so that the connector electrical contacts 42 are aligned with the device contacts 50. Although pin-type alignment structures are shown, other alignment structures may be employed, such as mated notches and tongues along the periphery of the clip surfaces. Also, the medical device 10 and clip 114 may be asymmetrically shaped such that device 10 insertion can be accomplished in only one orientation into clip 114.

In one embodiment, individual conductive silicone contacts are overmolded or post-inserted into the rigid clip 114 in place of a single piece of z-axis conductive elastomer 140. The individual contacts can also be co-molded into a single connector or subassembly part using a non-conductive elastomer or polymer to bridge the gap between each contact.

To improve electrical conductivity between the individual conductive elastic contacts and the patch, the bottom surface of the contacts may be printed or otherwise coated with conductive ink. In addition, a piece of Z-axis conductive adhesive may be laminated between the patch and the clip contacts, inside a window in the surrounding structural pressure sensitive adhesive

With continuing reference to FIG. 1 and further reference to FIGS. 16A and 16B, similar to the embodiments described above, the rigid clip 114 mechanically attaches the patch 22 to the monitoring device 10. The clip electrical contacts 42 include individual pieces or contacts 150 of the conductive elastomer, such as conductive silicone, which electrically connect the patch traces to the device contacts 50 on the bottom surface 52 of the monitoring device 10. Each individual conductive silicone piece 150 is surrounded by a elastomer seal 152. Such individual seals allow the individual pieces of silicon to maintain isolation from each other even if one seal fails.

In one embodiment, the elastomer contacts 150 are molded or inserted partway into the clip 114. The clip electrical contacts 42 further include silver/silver chloride (Ag/AgCl) plated plugs 154 which are insert-molded or post-inserted or applied into the bottom surface 40 of the clip 114 through openings 156 to make contact with each respective conductive elastomer contact 150 and the first hydrogel layer 28 of the patch 22.

As another example, the plug 154 is constructed from a conductive metal or plastic, such as a glass-fiber reinforced conductive acrylonitrile butadiene styrene (ABS), that is molded into shape. It is contemplated that the plug may or may not include a flange for touching the gel, and a post for press-fitting into the clip. The formed or molded plug is plated with the Ag/AgCl before or after molding.

As another example, the plug 154 can be die cut from a thin sheet of metal or conductive polymer, and then plated with the Ag/AgCl.

With continuing reference to FIG. 1 and further reference to FIGS. 17A and 17B, the clip 114 is similar to the clip of the embodiments described above, except the Ag/AgCl plugs 154 are omitted. The pieces 150 of the conductive elastomer material are overmolded through the entire thickness of the clip 114 so that the pieces 150 extend out or are flush with both the top and bottom surfaces 38, 40 of the clip 114. The exposed bottom surface 40 makes contact with metal traces or contacts of the patch 22, while the top surface 38 is exposed for contact with the device contacts 50 of the monitoring device 10.

In one embodiment, to create a half-cell reaction, the surface of each piece 150 of the elastomer material, such as silicone, is pad printed or screen-printed with a Ag/AgCl ink to make contact with the first conductive hydrogel layer 28 to form a half-cell reaction. To adequately adhere to the surface of the silicone pieces, the ink may be formulated in a silicone base.

In another embodiment, to create a half-cell reaction, the pieces of the conductive silicone are loaded with Ag/AgCl particles. E.g., the Ag/AgCl particles may be the conductive material in the silicone. If the loading is of high concentration, the Ag/AgCl particles in the cured silicone will form an adequate half-cell reaction as at the contact with the hydrogel.

With continuing reference to FIG. 1 and further reference to FIGS. 18A and 18B, the clip 114 includes clip openings 160. The clip electrical contacts 42 include rings 162 of Ag/AgCl which are pad or screen printed directly to the bottom surface 40 of the clip 114 to surround each clip opening 160. The clip electrical contacts 42 further include conductive silicone 164 which is overmolded completely through the openings 160 to overlap a portion of the printed Ag/AgCl rings 162 on the bottom surface 40 which contacts the patch 22. Upon contact with the first hydrogel layer 28, the rings 162 of Ag/AgCl create the half-cell reaction. The conductive silicone 164, which overlaps each printed ring 162 conducts the body signals to the contacts of the monitoring device 10.

In one embodiment, a vacuum is used to draw the Ag/AgCl ink inside the clip openings 160. In this design, the conductive silicone does not need to flow all the way through the clip opening 160 since the contact with the Ag/AgCl is made inside the clip opening 160.

With continuing reference to FIG. 1 and further reference to FIG. 19, the device contacts 50 include reusable conductive posts which are molded or inserted into the bottom housing of the monitoring device 10 to make direct contact with the hydrogel layer 28 of the patch assembly 14. For example, such posts are formed of metal or molded of conductive polymer. After forming, the posts are plated with Ag/AgCl before being inserted into the housing of the monitoring device 10. Since the posts can be cleaned between each application of the patch 22 to the patient, the Ag/AgCl coating should be thick and robust enough to withstand multiple cleanings and patch applications. Alternatively, the posts may be sintered out of Ag/AgCl. This eliminates plating the posts afterwards, and ensures that Ag/AgCl is not taken off the posts. The patch 22 may be mechanically attached to the monitoring device 10 in several ways.

With continuing reference to FIGS. 1 and 19 and further reference to FIG. 20, the patch 22 is attached to the monitoring device 10 with the clip 114, which is bonded to the patch 22 with a non-conductive PSA layer 165. Through the matching openings 166, 167 correspondingly provided in the clip 114 and the patch layers 24, the posts or device contacts 50 pass through the patch layers 24 and touch the first hydrogel layer 28 directly. Individual protective O-rings 168 are provided to seal and protect each post from liquid entry during bathing or showering.

As another example, the patch 22 can be attached to the monitoring device 10 via a non-conductive PSA layer, or a multi-layer PSA laminate. Openings are provided in the PSA layer to allow the Ag/AgCl posts to pass through and contact the first hydrogel layer 28. The PSA layer holds the patch 22 to the monitoring device 10 and seals around and between the individual posts. To ensure a consistent seal, a thicker PSA layer, or a thin (20 mil or 32-mil) PE or PU foam with adhesive on both sides may be used in place of the thick PSA. Being compressible, the foam compensates well for variations of Ag/AgCl post protrusion distances. In one embodiment, different adhesives on each side of the foam or PSA layer are used. More specifically, an aggressive PSA layer is used on the patch side and a less aggressive, easier to peal PSA layer, is used on the monitoring device side for the PSA material to come cleanly off of the monitoring device so that the monitoring device 10 can quickly be cleaned and prepared for use with a new patch.

The direct connection of the silver/silver chloride contacts with the hydrogel layer 28 eliminates conductive traces on the patch 22 and minimizes the number of connections required to make connection between the monitoring device and the base layer, improving reliability and potentially decreasing noise artifact. Without traces, the patch circuit layer 32 can become relatively inexpensive to manufacture. This also increases the material choices available for the circuit layer. For example, thinner polyester or PVDF films may be used if no printing is required. As the thinner films are used, the patch 22 becomes increasingly flexible and comfortable.

As mentioned above, Ag/AgCl is desirable as the hydrogel contact material for monitoring electrodes due to the stability of the resulting half-cell reaction.

With continuing reference to FIG. 1 and further reference to FIG. 21, the clip 114 is molded over a part of the patch circuit layer 32 via a technique similar to in-mold decorating techniques. More specifically, in-mold decorating techniques place a pre-formed printed polyester film into the injection mold against the inside surface of the mold. The molten polymer is then shot against the film and cooled. Once cooled, the polyester film is inseparable from the polymer. In one embodiment, the patch circuit layer 32 is a thin, printed polyester layer, suitable for placement in an injection-molding tool. As one example, the patch circuit 32 may be preformed into a shape. As another example, the patch circuit 32 may not be preformed into a shape. After insertion in the tool, the clip material is injected and cooled against its surface. This creates a strong mechanical bond between the patch circuit 32 and clip 114. The clip 114 includes the openings (not shown), which are positioned to communicate with the contact pads (not shown) in the circuit layer 32. The clip electrical connection interface 42 includes conductive silicone which is subsequently overmolded into the clip openings to form a robust electrical connection with the patch circuit 32. Non-conductive elastomeric rings may be molded or bonded around each conductive silicone contact for sealing against the surface of the monitoring device 10.

With continuing reference to FIG. 21 and further reference to FIGS. 22, 23, 24 and 25, the clip 114 and circuit sub-assembly is bonded to the patch 22. The foam layer or support layer 72 is coated on both sides with non-conductive PSA material. The PSA material connects and seals the patch circuit layer 32 and clip 114 to the foam layer 72. The foam layer 72 is attached to the hydrogel pieces 28 and retention seal 62.

FIG. 22 shows the patch circuit 32 proximate to the patch layers 24, with a patch side printed circuit 170.

FIG. 23 shows the patch circuit 32 proximate to the clip 114 with clip side printed contact pads 172.

FIG. 24 shows the clip 114 and the patch 22. The clip 114 includes clip openings 166 for the monitoring device posts or overmolding of conductive silicone contacts.

In one embodiment, the monitoring device 10 includes sensors 180 which detect body motion such as respirations, footfalls, heart beats and CPR compressions.

In this manner, by connecting the monitoring device to the medical patch via the thin clip helps to detect motion artifact in the monitored signal, which allows the signal processor in the monitoring device to compensate accordingly.

With reference again to FIG. 1, in one embodiment, the patch connector 26 is a low-profile electro-mechanical connection that creates a rigid patch area only in the center of the patch, leaving the outer areas of the patch flexible to bend and stretch with the skin. This minimizes the affect of monitor movement on the patch, thus reducing noise artifact due to monitor movement.

Other embodiments exist which include combinations of embodiments mentioned herein, combinations of low and higher-impedance contact mechanisms on the same connector, and embodiments that many include one, multiple or no seals.

The invention described above can be applied to other fields where electronic devices are attached to be held firmly to the skin to monitor physiologic signals or responses such as cardiac stress testing. One example is the athletic training field where electronic devices are worn to monitor performance. Other examples include child monitoring, such as for SIDS where a monitor is attached to the child for long periods of time to monitor cardiac and respiration activity, biosignal monitoring to monitor the health of the animals, and transdermal drug delivery systems which monitor certain patient parameters to determine when additional drug is required, how much is required, and the effects of drug dosage.

The methods and apparatuses described above provide a low-profile method of mechanically attaching the monitoring device to a thin, flexible patch. The thin, low-profile connections allow the monitoring device to become closely coupled to the bio-electrode patch enabling motion artifact detection by the monitoring device. When the electromechanical connection is thin enough, motion sensors in the monitoring device (accelerometers or piezo-electric sensors) can be designed to detect patient movements including footfalls, respirations and heartbeats.

The methods and apparatuses described above prevent the monitoring device from directly contacting the skin.

The methods and apparatuses described above include a low-profile, wire-free method of electrically connecting the patch electrodes to the monitoring device. Eliminating the wires can reduce electrical artifact in the bioelectric signal.

The methods and apparatuses described above include a method for creating a liquid-proof seal around each patch contact, or around the group of contacts, when the patch is connected to the monitoring device. Sealing between electrodes ensures that shorting does not occur during a shower or spill. The methods and apparatuses described can also be implemented without the seals.

The methods and apparatuses described above include a rigid central portion. Since the monitoring/therapy device is a relatively large mass attached to the skin, small movements or rotations in the device can create large disturbances in the monitored signal. A rigid portion in the center of the connector stabilizes the monitoring/therapy device and helps reduce motion artifact by preventing excess movement and rotation of the device during patient movement. When only the center is rigid, this connection scheme allows the edges of the patch to conform to body contours.

The methods and apparatuses described above are user friendly in that they may allow single step connections (both the mechanical and electrical connections are accomplished at the same time with the same user action). They can be performed with a single hand, and they do not require or transmit heavy forces to the body. They also allow attaching the monitoring device to the patch before attaching the patch to the skin.

The methods and apparatuses described above take advantage of the high-impedance patient monitoring electronics in the monitoring device. When impedances of the monitoring electronics are very high (i.e. Giga-ohm range), connector embodiments can be developed which are in the 1000-10,000 Ohm range without significantly affecting the monitored signal. The impedance attribute of the connector can be either high—100-10,000 Ohm, medium 20-200 Ohm, or low, <20 Ohm.

The invention has been described with reference to the preferred embodiments. Obviously, modifications and alterations will occur to others upon reading and understanding the preceding detailed description. It is intended that the invention be construed as including all such modifications and alterations insofar as they come within the scope of the appended claims or the equivalents thereof.

1. A skin-mounted device comprising:
a medical device which includes device electrical contacts;
a patch including a conductive first layer, which is in direct operative communication with the skin; and
a patch connector, which includes connector electrical contacts in electrical communication with the first conductive layer and establishes an electrical communication path between the skin and medical device contacts and which affixes the medical device in close proximity to the patch.

2. The device as set forth in claim 1, wherein the patch connector further includes:
alignment means for indexing the connector electrical contacts to match the medical device contacts.

3. The device as set forth in claim 1, wherein the patch connector includes:
a generally rigid clip including:
a clip first surfaces, and
a clip second surface
wherein the connector electrical contacts extend from about the clip first surface to about the clip second surface.

4. The device as set forth in claim 3, wherein the clip first surface includes extending portions which releasably engage a case of the medical device to hold the connector contacts and the device contacts in electrical communication.

5. The device as set forth in claim 3, further including:
a fluid seal which surrounds the connector contacts.

6. The device as set forth in claim 3, wherein the connector electrical contacts include at least one of:
pins;
snaps;
silver/silver chloride elements; and
electrically conductive elastic segments.

7. The device as set forth in claim 3, further including:
an electrical distribution layer which is electrically connected with the connector electrical contacts and with each of a plurality of first electrodes defined in the first conductive layer.

8. The device as set forth in claim 7, wherein the connector electrical contacts include:
a band of a z-axis conductive elastic material in contact with the electrical distribution layer.

9. The device as set forth in claim 1, wherein said medical device includes at least one of a cardiac ECG event monitor, ECG Holter recorder, and cardiac emergency alerting device.

10. The device as set forth in claim 1, further comprising:
a sensor which detects at least one parameter attributable to a motion of the skin.

11. The device as set forth in claim 1, wherein a thickness of the patch connector is less than 10 mm.

12. The device as set forth in claim 1, wherein the medical device electrical contacts are reusable.

13. A method of connecting a medical device to a subject base surface comprising:
disposing a patch which includes a conductive first layer in direct electrical communication with the base surface; and
electromechanically connecting the patch to a medical device connection interface which includes a first connector and device contacts via a patch connector.

14. The method as set forth in claim 13, wherein the patch layers further include second electrodes and further including:
disposing the first electrodes in direct electrical communication with the base surface and the second electrodes;
disposing the second electrodes in electrical communication with the device contacts; and
transmitting electrical signals generated in the base surface to the medical device contacts.

15. The method as set forth in claim 14, further including:
sealing the electrical communication path from the base surface to the device contacts against fluids with a sealing layer.

16. The method of claim 15, wherein the patch connector includes electrical contacts and the sealing layer includes elastic rings which each surrounds each respective individual patch electrical contact.

17. The method of claim 15, wherein the patch connector includes electrical contacts and the sealing layer includes an elastic material which encircles the patch electrical contacts.

18. The method as set forth in claim 13, wherein said connecting step occurs after said disposing step.

19. The method as set forth in claim 13, wherein the step of connecting includes:
detachably connecting the patch to the medical device connector via a clip which includes a first surface proximate the medical device and a second surface proximate the patch; and
simultaneously establishing the mechanical and electrical connections of the patch to the monitoring device.

20. The method as set forth in claim 19, wherein the clip includes:
a two-sided circuit layer with printed through-holes which electrically connect the first conductive layer which is disposed proximately to the clip second surface with traces disposed on the clip first surface.

21. The method as set forth in claim 19, wherein the clip includes clip contacts and further including:
mechanically snapping the medical device onto the clip so that the clip contacts are electrically connected with the first conductive layer on the clip second surface and the device contacts on the clip first surface to establish the electrical communication path between the base surface and the medical device contacts.

22. The method as set forth in claim 20, further including:
sealing the detachable connection between the clip contacts and the device contacts against fluids.

23. The method as set forth in claim 13, wherein the step of connecting includes:
establishing a rigid mechanical connection between the patch and the medical device.

24. A connector comprising:
a clip which includes:
a first surface;
a second surface opposite the first surface;
extending portions disposed on the first surface; and
a band of elastomeric material which extends from about the first surface to about the second surface to electrically contact device contacts of a medical device on the first surface and patch contacts on the second surface when the extending portions engage with a case of the medical device.
